(19) United States
(12) Patent Application Publication
(10) Pub. No.: US 2025/0003106 A1
(43) Pub. Date: Jan. 2, 2025

(54) FUNCTIONALIZING CARBON NANOSTRUCTURES

(71) Applicant: Canatu Oy, Vantaa (FI)

(72) Inventor: Ahmed Soliman, Vantaa (FI)

(21) Appl. No.: 18/739,830

(22) Filed: Jun. 11, 2024

(30) Foreign Application Priority Data
Jun. 28, 2023 (FI) 20235748

(51) Int. Cl. C25D 9/02; C23C 16/455

(52) U.S. Cl. CPC C25D 9/02; C23C 16/45525

(57) ABSTRACT

A method for producing a film of functionalized carbon nanostructures includes providing an electrode including a film of carbon nanostructures attached to a support, subjecting the electrode to an electrografting process in a bath containing water and at least one diazonium compound, conducting the electrografting process using potential pulses, wherein each potential pulse consists of an ON-time, wherein potential is applied for 0.01-0.1 s and an OFF-time, wherein zero potential is applied for 0.01-0.1 s, to form anchoring sites on the surfaces of the carbon nanostructures. Further is disclosed a film of functionalized carbon nanostructures. Further the use of the film or the method for forming a sensor, a filter, an electron stopping window, and/or a pellicle is explained.

### FIELD OF THE INVENTION

The present disclosure relates to a method for producing a film of functionalized carbon nanostructures attached to a support. The present disclosure further relates to a film of functionalized carbon nanostructures attached to a support. The present disclosure further relates to the use of the film of functionalized carbon nanostructures attached to a support for forming a sensor, a filter, an electron stopping window, and/or a pellicle. The present disclosure further relates to the use of the method as disclosed in the current specification for forming a sensor, a filter, an electron stopping window, and/or a pellicle.

### BACKGROUND OF THE INVENTION

Carbon nanostructures have desirable properties such as high surface area and good thermal and electrical conductivity. However, due to their inert nature, the surfaces of the carbon nanostructures may need to be functionalized in order to be suitable for further applications.

### SUMMARY OF THE INVENTION

A method for producing a film of functionalized carbon nanostructures attached to a support is disclosed. The method comprises:
providing an electrode comprising a film of carbon nanostructures attached to a support,
subjecting the electrode to an electrografting process in a bath containing water and at least one diazonium compound,
conducting the electrografting process using potential pulses, wherein each potential pulse consists of an ON-time, wherein potential is applied for 0.01-0.1 s, and an OFF-time, wherein zero potential is applied for 0.01-0.1 s, to form anchoring sites on the surfaces of the carbon nanostructures.

Further is disclosed a film of functionalized carbon nanostructures attached to a support, wherein the carbon nanostructures comprise anchoring sites on the surfaces of the carbon nanostructures, wherein each anchoring site is formed of a diazonium compound covalently bonded to the outer lateral surface of the carbon nanostructure.

Further is disclosed the use of the film of functionalized carbon nanostructures attached to a support as disclosed in the current specification for forming a sensor, a filter, an electron stopping window, and/or a pellicle.

Further is disclosed the use of the method as disclosed in the current specification for forming a sensor, a filter, an electron stopping window, and/or a pellicle.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 illustrates an FTIR spectrum of a film of functionalized carbon nanostructures according to one embodiment and an FTIR spectrum of a reference sample as described in example 1.

### DETAILED DESCRIPTION

A method for producing a film of functionalized carbon nanostructures attached to a support is disclosed. The method comprises:
providing an electrode comprising a film of carbon nanostructures attached to a support,
subjecting the electrode to an electrografting process in a bath containing water and at least one diazonium compound,
conducting the electrografting process using potential pulses, wherein each potential pulse consists of an ON-time, wherein potential is applied for 0.01-0.1 s, and an OFF-time, wherein zero potential is applied for 0.01-0.1 s, to form anchoring sites on the surfaces of the carbon nanostructures.

Further is disclosed a film of functionalized carbon nanostructures attached to a support, wherein the carbon nanostructures comprise anchoring sites on the surfaces of the carbon nanostructures, wherein each anchoring site is formed of a diazonium compound covalently bonded to the outer lateral surface of the carbon nanostructure.

Further is disclosed the use of the film of functionalized carbon nanostructures attached to a support as disclosed in the current specification for forming a sensor, a filter, an electron stopping window, and/or a pellicle. The film of functionalized carbon nanostructures attached to a support as disclosed in the current specification may be used as a sensor, a filter, an electron stopping window, and/or a pellicle.

Further is disclosed the use of the method as disclosed in the current specification for forming a sensor, a filter, an electron stopping window, and/or a pellicle.

In one embodiment, the sensor is an electrochemical sensor, a biosensor, or any combination thereof.

In one embodiment, the filter is an optical filter, a debris filter, a membrane filter, or any combination thereof. Thus, the filter may at the same time function as e.g. an optical filter and a debris filter. Thus, in one embodiment, the filter is an optical filter and a debris filter. In one embodiment, the filter is an optical filter and a membrane filter. In one embodiment, the filter is a debris filter and a membrane filter. In one embodiment, the filter is a optical filter, a debris filter, and a membrane filter.

An optical filter is a filter or device that that selectively transmits light or radiation of different wavelengths. An x-ray optical filter thus transmits x-ray radiation but may reject radiation of some different wavelength(s). Similarly, the EUV optical filter may transmit EUV radiation but may reject radiation of some different wavelength(s). An EUV debris filter then may transmit EUV radiation but may block debris and/or particles from passing through.

An electron stopping window is a device that transmits x-ray radiation and blocks electrons from transmitting through.

In one embodiment, the optical filter is an X-ray optical filter and/or an extreme ultraviolet (EUV) optical filter.

In one embodiment, the filter is an EUV debris filter and/or an EUV optical filter. In one embodiment, the filter is an EUV debris filter and an EUV optical filter. In one embodiment, the filter is an EUV debris filter or an EUV optical filter.

In one embodiment, the pellicle is an extreme ultraviolet lithography pellicle.

In one embodiment, the anchoring sites are used to immobilize and/or attach a biorecognition element. In one embodiment, the biorecognition element is an aptamer, an antibody, an antigen, an enzyme, a peptide, a cell, or a nucleic acid. The anchoring sites may be used to attach and/or immobilize a biorecognition element. The anchoring sites may function e.g. as an adapter so that the biorecognition element can be linked to the film of carbon nanostructures.

Various procedures exist in the art that may be used for forming the film of carbon nanostructures. Different manner may be used to synthesize the carbon nanostructures and/or to deposit the same to form a film. In the case of e.g. carbon nanotubes or carbon nanobud molecules, deposition may be carried out, for example, by using the commonly known methods of filtration from gas phase or from liquid, deposition in a force field, or deposition from a solution using spray coating or spin drying. The carbon nanobud molecules can be synthesized, for example, using the method disclosed in WO 2007/057501, and deposited on a substrate, for example, directly from the aerosol flow, e.g. by assistance of e.g. electrophoresis or thermophoresis, or by a method described in Nasibulin et al: “Multifunctional Free-Standing Single-Walled 20 Carbon Nanotube Films”, ACS NANO, vol. 5, no. 4, 3214-3221, 2011.

Functionalization of carbon nanostructures may be considered as the generation of functional groups on the surfaces of the carbon nanostructures. Functionalization of the carbon nanostructures has the added utility of making them more reactive, increasing their solubility, and/or allowing various chemical modifications, such as ion adsorption, metal deposition, and/or grafting reactions.

Electrografting is a process referring to an electrochemical reaction that permits organic layers and/or molecules to be attached to a solid conducting substrate, in this case the film of carbon nanostructures. In the method as disclosed in the current specification the diazonium compounds used in the electrografting process may form covalent bonds with the outer lateral surfaces of the carbon nanostructures. Thus, the expression “anchoring site” should be understood in this specification, unless otherwise stated, as a functional group formed by the diazonium compound covalently bonded to the lateral outer surface of the carbon nanostructure. The diazonium compound may react with the surface of the carbon nanostructure through its amino group. Thus, in one embodiment, the anchoring site comprises or consists of the diazonium compound covalently bonded to the surface of the carbon nanostructure through its amino group.

In potential pulse electrografting the potential may be alternated swiftly between two different values. This results in a series of pulses of equal amplitude, duration, and polarity, separated by zero current.

In the method as disclosed in the current specification the electrografting process thus comprises using potential pulses, wherein each potential pulse consists of an ON-time during which potential is applied, and an OFF-time during which zero potential is applied. In one embodiment, the electrografting process uses current pulses, wherein each current pulse consists of an ON-time during which current is applied, and an OFF-time during which zero current is applied. Thus, when controlling the potential, the current is let to follow, and wise versa.

In one embodiment, the applied potential is −800 mV to −600 mV, or −600 mV to −300 mV, or −300 My to −50 mV. In one embodiment, the potential is applied for 0.01-0.1 s, or 0.02-0.09 s, or 0.03-0.08 s, or 0.04-0.07 s, per each ON-time. In one embodiment, the zero potential is applied for 0.01-0.1 s, or 0.02-0.09 s, or 0.03-0.08 s, or 0.04-0.07 s, per each OFF-time.

The inventor surprisingly found out that when using the rather short pulsing times in the electrografting process, it is possible to minimize the evolution of hydrogen during the electrografting process. The hydrogen evolution may lead to uneven functionalization as the areas which are covered by hydrogen gas bubbles are not functionalized. The used short pulses used in the electrografting process enable the formation of anchoring sites, but the pulses are not long enough to lead to hydrogen evolution. Therefore, the method as disclosed in the current specification has the added utility of minimizing hydrogen evolution. Further, the hydrogen bubbles may appear as optical defects in the film of functionalized carbon nanostructures. Therefore, the method as disclosed in the current specification has the added utility of minimizing optical defects in the film of functionalized carbon nanostructures.

The electrografting process may be carried out for a total time of 1-5000 s, or 5-500 s, or 10-300 s, or 20-100 s, after which the electrografting process is stopped. In one embodiment, the electrografting process is carried out for a total time of 1-100 s, or 5-50 s, or 10-20 s.

The expression “nanostructures” should be understood in this specification, unless otherwise stated, as structures with one or more characteristic dimensions in nanometer scale, i.e. less or equal than about 100 nanometers. The dimensions of the conductive nanostructures, in two perpendicular directions, may be in significantly different magnitudes of order. For example, a nanostructure may have a length which is ten or hundred times higher than its thickness and/or width. In a film of carbon nanostructures, a great number of said carbon nanostructures are interconnected with each other to form a network of interconnected molecules. As considered at a macroscopic scale, such a network forms a solid, monolithic material in which the individual molecular structures are disoriented or non-oriented, i.e. are oriented substantially randomly, or oriented. Various types of carbon nanostructure networks can be produced in the form of thin transparent layers.

In one embodiment, the carbon nanostructures comprise carbon nanotubes (CNT), carbon nanobuds (CNB), carbon nanoribbons, or any combination thereof. In one embodiment, the carbon nanostructures comprise carbon nanotubes and/or carbon nanobuds. The carbon nanobuds, or the carbon nanobud molecules as they also may be called, have fullerene or fullerene-like molecules covalently bonded to the side of a tubular carbon molecule. In one embodiment, the carbon nanostructures are electrically conductive carbon nanostructures.

The support may be any type of support suitable to be attached with a film of carbon nanostructures. The support may be formed of a polymer, a metal, silicon, glass, a ceramic material, or any combination thereof.

The support may be provided with a current collector. A current collector may be used to collect electrons from the electrografting process. The current collector may typically be made of a conductive material, such as metal. The use of the current collector has the added utility of ensuring uniform deposition and controlling the deposition rate during the electrografting process.

In one embodiment, the film of (functionalized) carbon nanostructures is a free-standing film or a supported film. Thus, the form of the support may vary. The support may have the form of a frame. In one embodiment, the support has the form of a frame, and the film of (functionalized) carbon nanostructures is a free-standing film of (functionalized) carbon nanostructures attached to the frame. The frame may support the free-standing film of (functionalized) carbon nanostructures at the outer edges thereof such that an unsupported standalone region of the free-standing film of (functionalized) carbon nanostructures is formed. The support positions may be located anywhere in the structure as long as they provide sufficient support for the free-standing film of (functionalized) carbon nanostructures. For example, they may be on the sides of the free-standing film of (functionalized) carbon nanostructures, or in areas near corners, or next to each other along the sides. Any wider area that includes a plurality of support points is also meant to be covered by this aspect, for example if the frame has an uninterrupted circular shape wherein the free-standing region lies within the circle. The frame may also have any other prolonged uninterrupted shape. In one embodiment, the frame is shaped as a circle, a square, a triangle, a rectangle, an oval, or a polygon.

In one embodiment, the at least one diazonium compound is 1,10-phenanthrolin-5-amine, 6-amino-2-naphthoic acid, or 4′-amino-[1,1′-biphenyl]-4-carboxylic acid hydrochloride. I.e. the diazonium compound may be selected from a group consisting of 1,10-phenanthrolin-5-amine, 6-amino-2-naphthoic acid, and 4′-amino-[1,1′-biphenyl]-4-carboxylic acid hydrochloride. Also a combination or mixture of different diazonium compounds may be used. Thus, any combination of 1,10-phenanthrolin-5-amine, 6-amino-2-naphthoic acid, and 4′-amino-[1,1′-biphenyl]-4-carboxylic acid hydrochloride may be used.

The inventor surprisingly found out that these diazonium compounds have the added utility of improved EUV transmission as a result of having less oxygen. Further, these compounds have the added utility of molecular flexibility for attaching various sensor molecules such as biomolecules.

In one embodiment, the concentration of the diazonium compound in the bath is 0.1-100 mMol, or 1-90 mMol, or 3-80 mMol, or 5-70 mMol, or 10-60 mMol, or 15-50 mMol, or 20-40 mMol. The concentration of the diazonium compound in the bath may affect the density of the anchoring sites formed. If the concentration of the diazonium compound is too high, it may cause side reactions, whereby the compound starts to react with itself, i.e. to polymerize, instead of reacting with the carbon nanostructures. If the concentration of the diazonium compound in the bath is too low, it may not create enough anchoring sites i.e. surface concentration or density of the anchoring sites is too low.

In one embodiment, the bath further contains sulphuric acid and/or sodium nitride (NaNO₂). Sulphuric acid and/or sodium nitride in the bath may act as precursors and they may be used to in situ generate diazonium compound in the bath.

The film of (functionalized) carbon nanostructures may have the size of 0.1-10000 cm², or 1-7000 cm², or 10-5000 cm², or 100-3000 cm², or 500 2500 cm², or 1000-2000 cm². In one embodiment, the film of (functionalized) carbon nanostructures has the size of 0.1-1000 cm², or 1-500 cm², or 5-350 cm², or 10-200 cm², or 50-150 cm². The inventor surprisingly found out that with the method a disclosed in the current specification, one is able to functionalize a film of carbon nanostructures of even a large size that has not been possible before especially when the film of carbon nanostructures is a free-standing film of carbon nanostructures.

In one embodiment, the method further comprises forming a coating on the film of carbon nanostructures through the formed anchoring sites on the surfaces of the carbon nanostructures. In one embodiment, the coating is a nitride coating, a silicide coating, a coating of transition metal dichalcogenides, or a carbide coating. In one embodiment, the coating is a metal-based coating, such as a metal oxide coating.

In one embodiment, the coating is formed by an atomic layer deposition (ALD) type of process. The ALD process may be used to provide an ultra-thin conformal coating when applied in combination with the method as disclosed in the current specification. The formed anchoring sites on the surfaces of the carbon nanostructure enable the production of a coating on top of the film of carbon nanostructures.

The ALD-type of process is a method for depositing uniform and conformal deposits or layers over substrates, in this case over the film of carbon nanostructures, of various shapes, even over complex three-dimensional structures. In the ALD-type process, the substrate is alternately exposed to at least two different precursors (chemicals), usually one precursor at a time, to form on the substrate a deposit or a layer by alternately repeating essentially self-limiting surface reactions between the surface of the substrate (on the later stages, naturally, the surface of the already formed layer or deposit on the substrate) and the precursors. As a result, the deposited material is “grown” on the substrate molecule layer by molecule layer.

The distinctive feature of the ALD-type process is that the surface to be deposited is exposed to two or more different precursors in an alternate manner with usually a purging period in between the precursor pulses. During a purging period, the deposition surface is exposed to a flow of gas which does not react with the precursors used in the process. This gas, often called the carrier gas or the purge gas, is therefore inert towards the precursors used in the process and removes e.g. surplus precursor and by-products resulting from the chemisorption reactions of the previous precursor pulse. This purging can be arranged by different means.

Other names besides atomic layer deposition (ALD) have also been employed for these types of processes, where the alternate introduction of or exposure to two or more different precursors lead to the growth of the layer, often through essentially self-limiting surface reactions. These other names or process variants include atomic layer epitaxy (ALE), atomic layer chemical vapour deposition (ALCVD), and corresponding plasma enhanced, photo-assisted and electron enhanced variants. Unless otherwise stated, also these processes will be collectively addressed as ALD-type processes in this specification.

Forming a coating on the film of functionalized carbon nanostructures by the ALD-type process, has the added utility of one being able to use the anchoring sites for forming a passivation layer on the film. The method as disclosed in the current specification has the added utility of enabling the production of a uniform and large film of functionalized carbon nanostructures.

### EXAMPLES

Reference will now be made in detail to the described embodiments, examples of which are illustrated in the accompanying drawings.

The description below discloses some embodiments in such a detail that a person skilled in the art is able to utilize the method based on the disclosure. Not all steps of the embodiments are discussed in detail, as many of the steps will be obvious for the person skilled in the art based on this specification.

### Example 1—Producing a Film of Functionalizing Carbon Nanostructures Attached to a Support

In this example a film of carbon nanostructures was functionalized. Firstly, different electrodes were provided by attaching a preprepared film of carbon nanostructures to a support. The formed electrodes were then subjected to the electrografting process in an aqueous bath that was formed by using a diazonium compound, and then the electrografting process was carried out as described in the current specification.

The following materials and parameters were used in the examples:

| | Sample 1 | | Sample 2 | | Sample 3 | | Sample 4 | |
|---|---|---|---|---|---|---|---|---|
| Carbon | CNT | | CNT | | CNT | | CNT | |
| nanostructures | | | | | | | | |
| Material of the | PMMA | | Quartz | | PET | | Alumina | |
| support | | | | | | | | |
| Form of the | Frame | | Frame | | Frame | | Frame | |
| support | | | | | | | | |
| Area of the film of | 630 | cm² | 49 | cm² | 3600 | cm² | 1260 | cm² |
| carbon | | | | | | | | |
| nanostructures | | | | | | | | |
| Diazonium | 1,10- | | 6-amino-2- | | 4′-amino- | | 4′-amino- | |
| compound in the | phenanthrolin- | | naphthoic | | [1,1′- | | [1,1′- | |
| bath | 5-amine | | acid | | biphenyl]-4- | | biphenyl]-4- | |
| | | | | | carboxylic | | carboxylic | |
| | | | | | acid | | acid | |
| | | | | | hydrochloride | | hydrochloride | |
| Concentration of | 10 | mMol | 5 | mMol | 1 | mMol | 2 | mMol |
| the diazonium | | | | | | | | |
| compound in the | | | | | | | | |
| bath | | | | | | | | |
| Potential value | −50 | mV | −300 | mV | 800 | mV | 600 | mV |
| applied | | | | | | | | |
| Duration of ON- | 0.02 | s | 0.04 | s | 0.07 | s | 0.09 | s |
| time | | | | | | | | |
| Duration of OFF- | 0.09 | s | 0.04 | s | 0.08 | s | 0.02 | s |

time

CNT = carbon nanotube
PMMA = polymethylmethacrylate
PET = polyethylene terephthalate

The functionalization of sample 1 was confirmed through transmission Fourier-transform infrared (FTIR) measurements, which were conducted on both sample 1 and a reference sample. The reference sample represents the initial state before the method as described in the current specification was applied, i.e. a film of carbon nanostructures attached to a support that has not been functionalized. These measurements were carried out using Perkin Elmer spectrum3 device with 128 scans and resolution of 4 cm⁻¹.

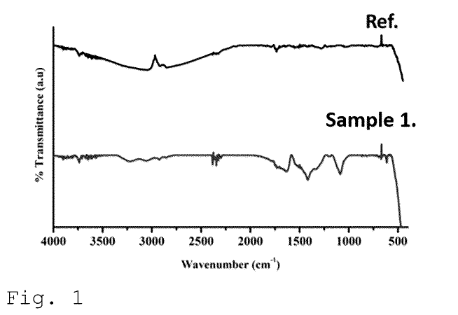

The functionalization of sample 1 was verified by comparing it with the reference sample. FIG. 1 shows the FTIR spectrum of sample 1 and the reference sample. In the spectrum of sample 1, the changes of the peaks compared to the spectrum of the reference sample correspond to the formed anchoring sites on the surfaces of the carbon nanostructures. FIG. 1 shows that the film of carbon nanostructures of sample 1 was uniformly functionalized.

From the above results one may see that one may functionalize carbon nanostructures by the applied method.

### Example 2—Coating the Film of Functionalized Carbon Nanostructures

In this example a silicon nitride coating was formed on top of the functionalized film of carbon nanostructures by using an ALD process. The following materials and parameters were used for making the coating:
Sample on which the coating was formed: Sample 1
Precursors used: SiCl₄ and NH₃
Deposition temperature: 500° C.
Deposition rate: 1.3 Å/cycle

The used precursors formed a co-ordinate bond with the free nitrogen/oxygen electron pairs of the 1,10-phenanthrolin-5-amine that has been covalently bonded to the outer lateral surface of the carbon nanotubes in sample 1. It was noted that a coating was efficiently formed on the film of functionalized carbon nanostructures of sample 1.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A method, a film of functionalized carbon nanostructures, or a use as disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A method for producing a film of functionalized carbon nanostructures attached to a support, wherein the method comprises:
providing an electrode comprising a film of carbon nanostructures attached to a support,
subjecting the electrode to an electrografting process in a bath containing water and at least one diazonium compound,
conducting the electrografting process using potential pulses, wherein each potential pulse consists of an ON-time, wherein potential is applied for 0.01-0.1 s, and an OFF-time, wherein zero potential is applied for 0.01-0.1 s, to form anchoring sites on the surfaces of the carbon nanostructures.

2. The method of claim 1, wherein each anchoring site is formed of the diazonium compound covalently bonded to the outer lateral surface of the carbon nanostructure.

3. The method of claim 1, wherein the at least one diazonium compound is 1,10-phenanthrolin-5-amine, 6-amino-2-naphthoic acid, or 4′-amino-[1,1′-biphenyl]-4-carboxylic acid hydrochloride.

4. The method of claim 1, wherein concentration of the diazonium compound in the bath is 0.1-100 mMol, or 1-90 mMol, or 3-80 mMol, or 5-70 mMol, or 10-60 mMol, or 15-50 mMol, or 20-40 mMol.

5. The method of claim 1, the bath further contains sulphuric acid and/or sodium nitride.

6. The method of claim 1, wherein the applied potential is −800 mV to −600 mV, or −600 mV to −300 mV, or −300 My to −50 mV.

7. The method of claim 1, wherein potential is applied for 0.02-0.09 s, or 0.03-0.08 s, or 0.04-0.07 s, per each ON-time.

8. The method of claim 1, wherein zero potential is applied for 0.02-0.09 s, or 0.03-0.08 s, or 0.04-0.07 s per each OFF-time.

9. The method of claim 1, wherein the film of carbon nanostructures has the size of 0.1-1000 cm2, or 1-500 cm2, or 5-350 cm2, or 10-200 cm2, or 50-150 cm2.

10. The method of claim 1, wherein the film of carbon nanostructures is a free-standing film or a supported film.

11. The method of claim 1, wherein support has the form of a frame, and the film of carbon nanostructures is a free-standing film of carbon nanostructures attached to the frame.

12. The method of claim 1, wherein the method further comprises forming a coating on the film of carbon nanostructures through the formed anchoring sites on the surfaces of the carbon nanostructures.

13. The method of claim 11, wherein the coating is formed by an atomic layer deposition (ALD) type of process.

14. A film of functionalized carbon nanostructures attached to a support, wherein the carbon nanostructures comprise anchoring sites on the surfaces of the carbon nanostructures, wherein each anchoring site is formed of a diazonium compound covalently bonded to the outer lateral surface of the carbon nanostructure.

15. The film of functionalized carbon nanostructures attached to a support of claim 14, wherein the diazonium compound is 1,10-phenanthrolin-5-amine, 6-amino-2-naphthoic acid, or 4′-amino-[1,1′-biphenyl]-4-carboxylic acid hydrochloride.

16. The film of functionalized carbon nanostructures attached to a support of claim 14, wherein the film of functionalized carbon nanostructures has the size of 0.1-1000 cm2, or 1-500 cm2, 5-350 cm2, or 10-200 cm2, or 50-150 cm2.

17. The film of functionalized carbon nanostructures attached to a support of claim 14, wherein the film of functionalized carbon nanostructures is a free-standing film or a supported film.

18. The film of functionalized carbon nanostructures attached to a support of claim 14, wherein support has the form of a frame, and the film of functionalized carbon nanostructures is a free-standing film of functionalized carbon nanostructures attached to the frame.

19. The film of functionalized carbon nanostructures attached to a support of claim 14, wherein a coating is formed on the film of functionalized carbon nanostructures through the anchoring sites on the surfaces of the carbon nanostructures.

20. The use of the method of claim 1, for forming a sensor, a filter, an electron stopping window, and/or a pellicle.

21. The use of the film of functionalized carbon nanostructures attached to a support of claim 14, for forming a sensor, a filter, an electron stopping window, and/or a pellicle.

22. The use of claim 20, wherein the sensor is an electrochemical sensor, a biosensor, or any combination thereof.

23. The use of claim 20, wherein the filter is an optical filter, a debris filter, a membrane filter, or any combination thereof.

24. The use of claim 20, wherein the filter is an optical filter and a debris filter.

25. The use of claim 23, wherein the optical filter is an X-ray optical filter, an EUV optical filter, or any combination thereof.

26. The use of claim 20, wherein the pellicle is an extreme ultraviolet lithography pellicle.

27. The use of claim 20, wherein the anchoring sites are used to immobilize and/or attach a biorecognition element.

28. The use of claim 27, wherein the biorecognition element is an aptamer, an antibody, an antigen, an enzyme, a peptide, a cell, or a nucleic acid.
